(19) United States
(12) Patent Application Publication
(10) Pub. No.: US 2006/0116360 A1
(43) Pub. Date: Jun. 1, 2006

(54) Method of administering anti-angiogenic agents and a method of treating disease using same

(76) Inventors: William E. Fogler, Rockville, MD (US); Carolyn Sidor, Chapel Hill, NC (US); Anthony M. Treston, Rockville, MD (US); Kirk M. Volker, North Potomac, MD (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/288,989

(22) Filed: Nov. 29, 2005

Related U.S. Application Data
(60) Provisional application No. 60/631,502, filed on Nov. 29, 2004.
(60) Provisional application No. 60/715,238, filed on Sep. 8, 2005.
(60) Provisional application No. 60/732,065, filed on Nov. 1, 2005.

(51) Int. Cl. A61K 31/56

(52) U.S. Cl. 514/182

(57) ABSTRACT

A method of administering an anti-angiogenic agent to a human or an animal comprising administering the anti-angiogenic agent such that a plasma concentration of the anti-angiogenic agent in the human or animal is substantially continuously maintained above 1 ng/mL.

### CROSS-REFERENCE TO RELATED APPLICATIONS

The present application claims the benefit of U.S. Provisional Patent Application Ser. No. 60/631,502, filed Nov. 29, 2004; U.S. Provisional Patent Application Ser. No. 60/715,238, filed Sep. 8, 2005; and U.S. Provisional Patent Application Ser. No. 60/732,065, filed Nov. 1, 2005.

### FIELD OF THE INVENTION

The present invention relates generally to a method of administering anti-angiogenic agents. More specifically, the present invention relates to a method of administering 2-methoxyestradiol, or analogs of 2-methoxyestradiol, such that a desired plasma concentration is substantially continuously maintained above predetermined blood concentrations. More particularly, the present invention relates to a method of treating diseases characterized by abnormal cell mitosis and/or abnormal or undesirable angiogenesis by administering 2-methoxyestradiol, or analogs of 2-methoxyestradiol, such that a desired plasma concentration is substantially continuously maintained.

### BACKGROUND OF THE INVENTION

Angiogenesis is the generation of new blood vessels into a tissue or organ. Under normal physiological conditions, humans and animals undergo angiogenesis only in very specific, restricted situations. For example, angiogenesis is normally observed in wound healing, fetal and embryonal development, and formation of the corpus luteum, endometrium and placenta.

Angiogenesis is controlled through a highly regulated system of angiogenic stimulators and inhibitors. The control of angiogenesis has been found to be altered in certain disease states and, in many cases, pathological damage associated with the diseases is related to uncontrolled angiogenesis. Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. Endothelial cells, lining the lumen of blood vessels, then protrude through the basement membrane. Angiogenic stimulants induce the endothelial cells to migrate through the eroded basement membrane. The migrating cells form a “sprout” off the parent blood vessel where the endothelial cells undergo mitosis and proliferate. The endothelial sprouts merge with each other to form capillary loops, creating a new blood vessel.

Persistent, unregulated angiogenesis occurs in many disease states, tumor metastases, and abnormal growth by endothelial cells. The diverse pathological disease states in which unregulated angiogenesis is present have been grouped together as angiogenic-dependent or angiogenic-associated diseases.

One example of a disease dependent on angiogenesis is ocular neovascular disease. This disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma, and retrolental fibroplasia. Other diseases associated with corneal neovascularization include, but are not limited to, epidemic keratoconjunctivitis, Vitamin A deficiency, contact lens overwear, atopic keratitis, superior limbic keratitis, and pterygium keratitis sicca. Other diseases associated with undesirable angiogenesis include Sjögren's syndrome, acne rosacea, phylectenulosis, syphilis, Mycobacterial infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, rheumatoid arthritis, systemic lupus, polyarteritis, trauma, Wegener's sarcoidosis, scleritis, Stevens-Johnson's disease, pemphigoid, and radial keratotomy.

Diseases associated with neovascularization include, but are not limited to, retinal/choroidal neovascularization, diabetic retinopathy, macular degeneration, sickle cell anemia, sarcoidosis, syphilis, pseudoxanthoma elasticum, Paget's disease, vein occlusion, artery occlusion, carotid obstructive disease, chronic uveitis/vitritis, Mycobacterial infections, Lyme's disease, systemic lupus erythematosis, retinopathy of prematurity, Eales' disease, Behcet's disease, infections causing retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other eye-related diseases include, but are not limited to, diseases associated with rubeosis (neovascularization of the iris and of the angle) and diseases caused by the abnormal proliferation of fibrovascular or fibrous tissue, including all forms of prolific vitreoretinopathy.

Another angiogenesis associated disease is rheumatoid arthritis. The blood vessels in the synovial lining of the joints undergo angiogenesis. In addition to forming new vascular networks, the endothelial cells release factors and reactive oxygen species that lead to pannus growth and cartilage destruction. Angiogenesis may also play a role in osteoarthritis. The activation of the chondrocytes by angiogenic-related factors contributes to the destruction of the joint. At a later stage, the angiogenic factors promote new bone growth. Therapeutic intervention that prevents the cartilage destruction could halt the progress of the disease and provide relief for persons suffering with arthritis.

Chronic inflammation may also involve pathological angiogenesis. Such diseases as ulcerative colitis and Crohn's disease show histological changes with the ingrowth of new blood vessels into inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity.

The hypothesis that tumor growth is angiogenesis-dependent was first proposed in 1971. (Folkman, New Eng. J Med., 285:1182-86 (1971)). In its simplest terms, this hypothesis states: “Once tumor ‘take’ has occurred, every increase in tumor cell population must be preceded by an increase in new capillaries converging on the tumor.” Tumor ‘take’ is currently understood to indicate a prevascular phase of tumor growth in which a population of tumor cells occupying a few cubic millimeters volume, and not exceeding a few million cells, can survive on existing host microvessels. Expansion of tumor volume beyond this phase requires the induction of new capillary blood vessels. For example, pulmonary micrometastases in the early prevascular phase in mice would be undetectable except by high power microscopy on histological sections.

Examples of the indirect evidence which support this concept include:

(1) The growth rate of tumors implanted in subcutaneous transparent chambers in mice is slow and linear before neovascularization, and rapid and nearly exponential after neovascularization. (Algire, et al., J. Nat. Cancer Inst., 6:73-85 (1945)).

(2) Tumors grown in isolated perfused organs where blood vessels do not proliferate are limited to 1-2 mm³ but expand rapidly to >1000 times this volume when they are transplanted to mice and become neovascularized. (Folkman, et al., Annals of Surgery, 164:491-502 (1966)).

(3) Tumor growth in the avascular cornea proceeds slowly and at a linear rate, but switches to exponential growth after neovascularization. (Gimbrone, Jr., et al., J. Nat. Cancer Inst., 52:421-27 (1974)).

(4) Tumors suspended in the aqueous fluid of the anterior chamber of a rabbit eye remain viable, avascular, and limited in size to <1 mm³. Once they are implanted on the iris vascular bed, they become neovascularized and grow rapidly, reaching 16,000 times their original volume within 2 weeks. (Gimbrone, Jr., et al., J. Exp. Med., 136:261-76).

(5) When tumors are implanted on a chick embryo chorioallantoic membrane, they grow slowly during an avascular phase of >72 hours, but do not exceed a mean diameter of 0.93+0.29 mm. Rapid tumor expansion occurs within 24 hours after the onset of neovascularization, and by day 7 these vascularized tumors reach a mean diameter of 8.0+2.5 mm. (Knighton, British J. Cancer, 35:347-56 (1977)).

(6) Vascular casts of metastases in a rabbit liver reveal heterogeneity in size of the metastases, but show a relatively uniform cut-off point for the size at which vascularization is present. Tumors are generally avascular up to 1 mm in diameter, but are neovascularized beyond that diameter. (Lien, et al., Surgery, 68:334-40 (1970)).

(7) In transgenic mice that develop carcinomas in the beta cells of the pancreatic islets, pre-vascular hyperplastic islets are limited in size to <1 mm. At 6-7 weeks of age, 4-10% of the islets become neovascularized, and from these islets arise large vascularized tumors of more than 1000 times the volume of the pre-vascular islets. (Folkman, et al., Nature, 339:58-61 (1989)).

(8) A specific antibody against VEGF (vascular endothelial growth factor) reduces microvessel density and causes “significant or dramatic” inhibition of growth of three human tumors which rely on VEGF as their sole mediator of angiogenesis (in nude mice). The antibody does not inhibit growth of the tumor cells in vitro. (Kim, et al., Nature, 362:841-44 (1993)).

(9) Anti-bFGF monoclonal antibody causes 70% inhibition of growth of a mouse tumor which is dependent upon secretion of bFGF as its only mediator of angiogenesis. The antibody does not inhibit growth of the tumor cells in vitro. (Hori, et al., Cancer Res., 51:6180-84 (1991)).

(10) Intraperitoneal injection of bFGF enhances growth of a primary tumor and its metastases by stimulating growth of capillary endothelial cells in the tumor. The tumor cells themselves lack receptors for bFGF, and bFGF is not a mitogen for the tumor cells in vitro. (Gross, et al., Proc. Am. Assoc. Cancer Res., 31:79 (1990)).

(11) A specific angiogenesis inhibitor (AGM-1470) inhibits tumor growth and metastases in vivo, but is much less active in inhibiting tumor cell proliferation in vitro. It inhibits vascular endothelial cell proliferation half-maximally at 4 logs lower concentration than it inhibits tumor cell proliferation. (Ingber, et al., Nature, 48:555-57 (1990)). There is also indirect clinical evidence that tumor growth is angiogenesis dependent.

(12) Human retinoblastomas that are metastatic to the vitreous develop into avascular spheroids that are restricted to less than 1 mm³ despite the fact that they are viable and incorporate ³H-thymidine (when removed from an enucleated eye and analyzed in vitro).

(13) Carcinoma of the ovary metastasizes to the peritoneal membrane as tiny avascular white seeds (1-3 mm³). These implants rarely grow larger until one or more of them become neovascularized.

(14) Intensity of neovascularization in breast cancer (Weidner, et al., New Eng. J. Med., 324:1-8(1991); Weidner, et al, J Nat. Cancer Inst., 84:1875-87 (1992)) and in prostate cancer (Weidner, et al., Am. J. Pathol., 143(2):401-09 (1993)) correlates highly with risk of future metastasis.

(15) Metastasis from human cutaneous melanoma is rare prior to neovascularization. The onset of neovascularization leads to increased thickness of the lesion and an increased risk of metastasis. (Srivastava, et al., Am. J. Pathol., 133:419-23 (1988)).

(16) In bladder cancer, the urinary level of an angiogenic protein, bFGF, is a more sensitive indicator of status and extent of disease than is cytology. (Nguyen, et al., J. Nat. Cancer Inst., 85:24142 (1993)).

Thus, it is clear that angiogenesis plays a major role in the metastasis of cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

Angiogenesis has been associated with a number of different types of cancer, including solid tumors and blood-borne tumors. Solid tumors with which angiogenesis has been associated include, but are not limited to, rhabdomyosarcomas, retinoblastoma, Ewing's sarcoma, neuroblastoma, and osteosarcoma. Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed that angiogenesis plays a role in the abnormalities in the bone marrow that give rise to leukemia tumors and multiple myeloma diseases.

One of the most frequent angiogenic diseases of childhood is the hemangioma. A hemangioma is a tumor composed of newly formed blood vessels. In most cases the tumors are benign and regress without intervention. In more severe cases, the tumors progress to large cavernous and infiltrative forms and create clinical complications. Systemic forms of hemangiomas, hemangiomatoses, have a high mortality rate. Therapy-resistant hemangiomas exist that cannot be treated with therapeutics currently in use.

Angiogenesis is also responsible for damage found in heredity diseases such as Osler-Weber-Rendu disease, or heredity hemorrhagic telangiectasia. This is an inherited disease characterized by multiple small angiomas, tumors of blood or lymph vessels. The angiomas are found in the skin and mucous membranes, often accompanied by epitaxis (nose bleeds) or gastrointestinal bleeding and sometimes with pulmonary or hepatitic arteriovenous fistula.

What is needed, therefore, is a composition and method that can inhibit angiogenesis. What is also needed is a composition and method that can inhibit the unwanted growth of blood vessels, especially in tumors.

Angiogenesis is also involved in normal physiological processes, such as reproduction and wound healing. Angiogenesis is an important step in ovulation and also in implantation of the blastula after fertilization. Prevention of angiogenesis could be used to induce amenorrhea, to block ovulation, or to prevent implantation by the blastula.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

Several compounds have been used to inhibit angiogenesis. Taylor, et al. (Nature, 297:307 (1982)) have used protamine to inhibit angiogenesis. The toxicity of protamine limits its practical use as a therapeutic. Folkman, et al. (Science, 221:719 (1983), and U.S. Pat. Nos. 5,001,116 and 4,994,443) have disclosed the use of heparin and steroids to control angiogenesis. Steroids, such as tetrahydrocortisol, which lack glucocorticoid and mineralocorticoid activity, have been found to be angiogenic inhibitors.

Other factors found endogenously in animals, such as a 4 kDa glycoprotein from bovine vitreous humor and a cartilage derived factor, have been used to inhibit angiogenesis. Cellular factors, such as interferon, inhibit angiogenesis. For example, interferon alpha or human interferon beta have been shown to inhibit tumor-induced angiogenesis in mouse dermis stimulated by human neoplastic cells. Interferon beta is also a potent inhibitor of angiogenesis induced by allogeneic spleen cells. (Sidky, et al., Cancer Res., 47:5155-61(1987)). Human recombinant interferon (alpha/A) was reported to be successfully used in the treatment of pulmonary hemangiomatosis, an angiogenesis-induced disease. (White, et al., New Eng. J. Med., 320:1197-1200 (1989)).

Other agents that have been used to inhibit angiogenesis include ascorbic acid ethers and related compounds. (Japanese Kokai Tokkyo Koho No.58-13 (1978)). Sulfated polysaccharide DS 4152 also inhibits angiogenesis. (Japanese Kokai Tokkyo Koho No. 63-119500). Additional anti-angiogenic compounds include Angiostatin® (U.S. Pat. Nos. 5,639,725; 5,792,845; 5,885,795; 5,733,876; 5,776,704; 5,837,682; 5,861,372, and 5,854,221, the disclosures of which are all incorporated herein by reference) and Endostatin (U.S. Pat. No. 5,854,205, the disclosure of which is incorporated herein by reference).

Another compound which has been shown to inhibit angiogenesis is thalidomide. (D'Amato, et al., Proc. Natl. Acad. Sci., 90:4082-85 (1994)). Thalidomide is a hypnosedative that has been successfully used to treat a number of diseases, such as rheumatoid arthritis (Gutierrez-Rodriguez, Arthritis Rheum., 27 (10):1118-21 (1984); Gutierrez-Rodriguez, et al., J. Rheumatol., 16(2):158-63 (1989)), and Behcet's disease (Handley, et al., Br. J. Dermatol., 127 Suppl, 40:67-8 (1992); Gunzler, Med. Hypotheses, 30(2):105-9 (1989)).

Although thalidomide has minimal side effects in adults, it is a potent teratogen. Thus, there are concerns regarding its use in women of child-bearing age. Although minimal, there are a number of side effects that limit the desirability of thalidomide as a treatment. One such side effect is drowsiness. In a number of therapeutic studies, the initial dosage of thalidomide had to be reduced because patients became lethargic and had difficulty functioning normally. Another side effect limiting the use of thalidomide is peripheral neuropathy, in which individuals suffer from numbness and dysfunction in their extremities.

Heretofore, it has not been known what is the most effective method of administering anti-angiogenic agents to humans or animals to inhibit angiogenesis and/or to treat diseases or conditions associated with angiogenesis. Furthermore, heretofore it has not been known what dose and what dosing schedule is the most effective method of administering anti-angiogenic agents to humans or animals to inhibit angiogenesis and/or to treat diseases or conditions associated with or dependent on angiogenesis. Therefore, there is a great need for a method, dose and schedule for administering anti-angiogenic agents to humans or animals to most effectively inhibit angiogenesis and/or to treat diseases or conditions associated with or dependent on undesirable and/or excessive angiogenesis and/or undesirable cell mitosis.

### SUMMARY OF THE INVENTION

The present invention satisfies the foregoing need by providing a method of administering an anti-angiogenic agent to a human or an animal comprising administering the anti-angiogenic agent such that a plasma concentration of the anti-angiogenic agent in the human or animal is substantially continuously maintained above approximately 1 ng/mL. Preferably, the plasma concentration of the agent is between approximately 1 ng/mL and 300 ng/mL, more preferably between approximately 2 ng/mL and 150 ng/mL and most preferably between approximately 3 ng/mL and 100 ng/mL. The most preferable plasma concentration range is between approximately 3 and 50 ng/mL.

In an alternate embodiment, the present invention comprises a method of administering analogs and/or derivatives of estrogen to a human or an animal comprising administering the analogs and/or derivatives of estrogen such that a plasma concentration of the analogs and/or derivatives of estrogen in the human or animal is substantially continuously maintained at or above approximately 3 to approximately 50 ng/mL.

In a further embodiment, the present invention comprises a method of administering 2-methoxyestradiol and analogs and/or derivatives of 2-methoxyestradiol to a human or an animal, comprising administering the 2-methoxyestradiol and analogs and/or derivatives of 2-methoxyestradiol, such that a plasma concentration of the 2-methoxyestradiol and analogs and/or derivatives of 2-methoxyestradiol in the human or animal is substantially continuously maintained above approximately 3 ng/mL. Preferably the plasma concentration of the agent is between approximately 1 ng/mL and 300 ng/mL, more preferably between approximately 2ng/mL and 150 ng/mL and most preferably between approximately 3 ng/mL and 100 ng/mL. The most preferable plasma concentration range is between approximately 3 and 50 ng/mL.

In another embodiment, the present invention comprises a method of treating disease in a human or animal comprising administering to the human or animal having the disease an amount of a therapeutic agent selected from 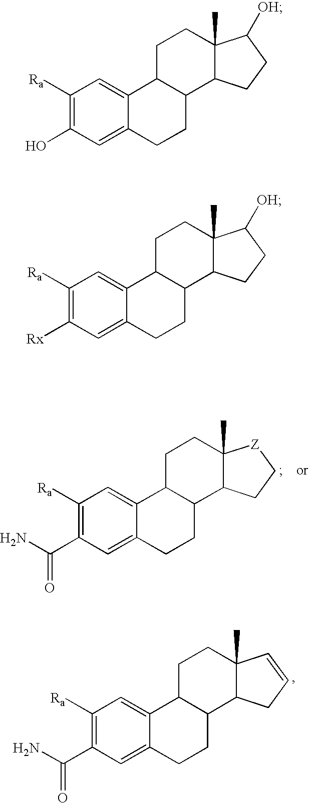
wherein Rₐ is selected from —OCH₃, —OCH₂CH₃, —CH₃, —CH₂CH₃, or —CCCH₃; and Rₓ is selected from —NH₂, —F, —Cl, —Br, —CH═CH₂, —NH—CHO, —O-sulfamate; and Z is selected from >C(H₂), >C(H)—CH₃, >C═CH₂, >C═CHCH₃ (cis or trans), >C═O, >C(H)—OH, >C(H)—O-alkyl, or >C(H)—O-sulfamate, wherein alkyl is a linear, branched and/or cyclic hydrocarbon chain comprising 1 to 10 carbons, such that a plasma concentration of the anti-angiogenic agent in the human or animal is substantially continuously maintained above approximately 1 ng/mL. Preferably the plasma concentration of the agent is between approximately 1 ng/mL and 300 ng/mL, more preferably between approximately 2 ng/mL and 150 ng/mL and most preferably between approximately 3 ng/mL and 100 ng/mL. The most preferable plasma concentration range is between approximately 3 and 50 ng/mL.

In another embodiment, the present invention comprises a method of treating diseases or conditions associated with or dependent on abnormal, undesirable and/or excessive angiogenesis and/or undesirable cell mitosis in a human or animal comprising administering to the human or animal an amount of an anti-angiogenic agent selected from 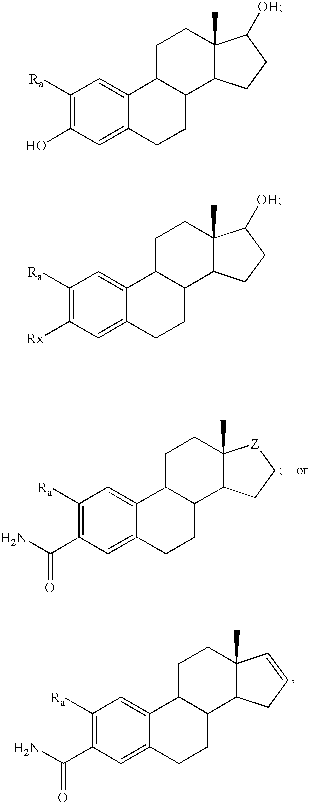
wherein Rₐ is selected from —OCH₃, —OCH₂CH₃, —CH₃, —CH₂CH₃, or —CCCH₃; and Rₓ is selected from —NH₂, —F, —Cl, —Br, —CH═CH₂, —NH—CHO, —O-sulfamate; and Z is selected from >C(H₂), >C(H)—CH₃, >C═CH₂, C═CHCH₃ (cis or trans), >C═O, >C(H)—OH, >C(H)—O-alkyl, or >C(H)—O-sulfamate, wherein alkyl is a linear, branched and/or cyclic hydrocarbon chain comprising 1 to 10 carbons, such that a plasma concentration of the anti-angiogenic agent in the human or animal is substantially continuously maintained at approximately 3 to approximately 50 ng/mL. Preferably the plasma concentration of the agent is between approximately 1 ng/mL and 300 ng/mL, more preferably between approximately 2ng/mL and 150 ng/mL and most preferably between approximately 3 ng/mL and 100 ng/mL. The most preferable plasma concentration range is between approximately 3 and 50 ng/mL.

In yet another embodiment, the present invention comprises a pharmaceutical preparation comprising a compound selected from 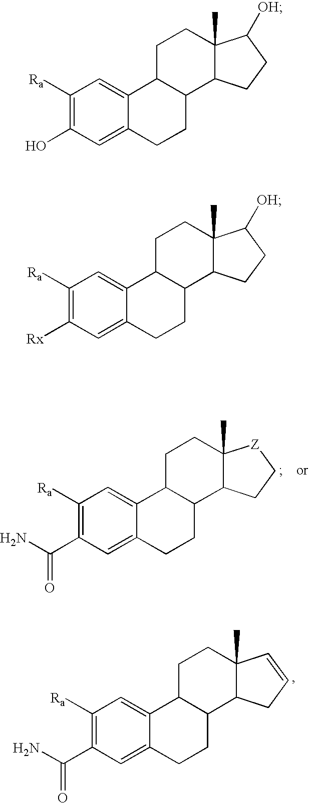
wherein Rₐ is selected from —OCH₃, —OCH₂CH₃, —CH₃, —CH₂CH₃, or —CCCH₃; and Rₓ is selected from —NH₂, —F, —Cl, —Br, —CH═CH₂, —NH—CHO, —O-sulfamate; and Z is selected from >C(H₂), >C(H)—CH₃, >C═CH₂, >C═CHCH₃ (cis or trans), >C═O, >C(H)—OH, >C(H)—O-alkyl, or >C(H)—O-sulfamate, wherein alkyl is a linear, branched and/or cyclic hydrocarbon chain comprising 1 to 10 carbons. The amount of the compound in the pharmaceutical preparation and the dosing schedule are chosen such that when the pharmaceutical preparation is administered to a human or an animal a plasma concentration of the anti-angiogenic agent in the human or animal is substantially continuously maintained at or above approximately 3 to approximately 50 ng/mL. Preferably the plasma concentration of the agent is between approximately 1 ng/mL and 300 ng/mL, more preferably between approximately 2 g/mL and 150 ng/mL and most preferably between approximately 3 ng/mL and 100 ng/mL. The most preferable plasma concentration range is between approximately 3 and 50 ng/mL. The pharmaceutical preparation can also comprise a pharmaceutically acceptable carrier, excipient or diluent.

Accordingly, it is an object of the present invention to provide a method of administering an anti-angiogenic agent to a human or an animal.

Another object of the present invention is to provide a method of administering an anti-angiogenic agent to a human or an animal to maintain a desired plasma concentration of the anti-angiogenic agent in the human or an animal substantially continuously.

A further object of the present invention is to provide a method, dose and schedule for administering anti-angiogenic agents to mammals to more effectively inhibit angiogenesis and/or to more effectively treat diseases or conditions associated with undesirable and/or excessive angiogenesis and/or undesirable cell mitosis.

These and other objects, features and advantages of the present invention will become apparent after a review of the following detailed description of disclosed embodiments and the appended claims.

### BRIEF DESCRIPTION OF THE DRAWINGS

FIG. 1 shows the effects of transiently high levels of 2-methoxyestradiol (2ME2).

FIG. 2 shows plasma blood levels of 2ME2.

FIG. 3A shows inhibition of pulmonary metastases.

FIG. 3B shows plasma levels of 2ME2 after implant.

FIGS. 4A and 4B show 2ME2 concentration after a series of wash-out anti-proliferation studies.

FIGS. 5A and 5B show inhibition of pulmonary metastases in tumor bearing mice given 2ME2 ad libitum.

FIG. 6 is shows the effect of cyclic levels of ENMD-1198 compared to the effects of continuous exposure of ENMD-1198.

### DETAILED DESCRIPTION OF THE INVENTION

The present invention comprises a method of administering an anti-angiogenic agent to a human or an animal comprising administering the anti-angiogenic agent such that a plasma concentration of the anti-angiogenic agent in the human or animal is substantially continuously maintained at or above approximately 1 ng/mL. Preferably the plasma concentration of the agent is between approximately 1 ng/mL and 300 ng/mL, more preferably between approximately 2 ng/mL and 150 ng/mL and most preferably between approximately 3 ng/mL and 100 ng/mL. The most preferable plasma concentration range is between approximately 3 and 50 ng/mL. As used herein the term “substantially continuously” means during more than 70% of the time in each 24 hour period. Preferably, the desired plasma concentration of the anti-angiogenic agent is maintained during more than 80% of the time in each 24 hour period; more preferably, more than 90% of the time in each 24 hour period; especially, 100% of the time in each 24 hour period.

As described below, anti-angiogenic agents are those compounds that exhibit anti-angiogenesis, anti-inflammatory, anti-mitotic and/or anti-tumor activity in humans and animals. Preferred agents are analogs and/or derivatives of estrogen that exhibit anti-angiogenesis, anti-mitotic and/or anti-tumor activity in humans and animals. Particularly preferred compounds are 2-methoxyestradiol and analogs of 2-methoxyestradiol that exhibit anti-mitotic, anti-angiogenic, anti-inflammatory and/or anti-tumor properties. Especially preferred compounds of the invention are 2-methoxyestradiol analogs and/or derivatives modified at the 2-, 3- or 17-positions or at combinations of the 2-, 3-, and 17-positions. Most preferred compounds are those of the general Formulae I, II, III or IV: 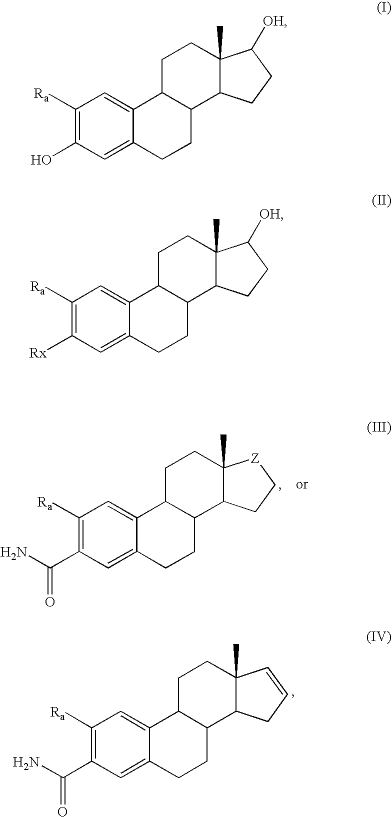
wherein Rₐ is selected from —OCH₃, —OCH₂CH₃, —CH₃, —CH₂CH₃, or —CCCH₃; and Rₓ is selected from —NH₂, —F, —Cl, —Br, —CH═CH₂, —NH—CHO, —O-sulfamate; and Z is selected from >C(H₂), >C(H)—CH₃, >C═CH₂, >C═CHCH₃ (cis or trans), >C═O, >C(H)—OH, >C(H)—O-alkyl or >C(H)—O-sulfamate. Alkyl is defined herein as a linear, branched and/or cyclic hydrocarbon chain containing 1-10 carbons. Preferred species according to the present invention are described below.

Also included in this invention are prodrugs of the anti-angiogenic agents. Such prodrugs are produced using chemical procedures and synthetic routes well known to those of ordinary skill in the art. Strategies for creating prodrugs are well known to those skilled in the art and include amides, esters, ethers, thioesters, and thioethers of the agent. The chemical moieties linked to the agent can include naturally and non-naturally occurring amino acids, sugars, peptides, low molecular weight organic moieties such as acetate and carbamate and benzoate and benzoyl, proteins including antibodies, and polymers including, but not limited to, polyethyleneglycols (PEGs). These moieties can be cleaved by enzymatic or non-enzymatic processes to generate the active anti-angiogenic agent. The characteristics of the prodrug are chosen to be useful for the desired purposes and routes of administration. For examples, prodrugs containing amino acids can be used to enhance water solubility, prodrugs containing peptides can be used to enhance bioavailability and/or to enhance target tissue binding, and prodrugs containing PEGs can be used to increase plasma half-life.

Other anti-angiogenic agents useful in the present invention are disclosed in U.S. Pat. Nos. 5,504,074; 6,605,622; and U.S. patent application Ser. Nos. 09/641,327, filed Aug. 18, 2000; 60/255,302, filed Dec. 13, 2000; Ser. No. 09/779,331, filed Feb. 8, 2001; 60/278,250, filed Mar. 23, 2001; Ser. No. 09/939,208, filed Aug. 24, 2001; 60/354,046, filed Jan. 30, 2002; Ser. No. 10/354,927, filed Jan. 30, 2003; Ser. No. 10/354,921, filed Jan. 30, 2003; 60/474,288, filed May 28, 2003; 60/552,692, filed Mar. 12, 2004; 60/562,793, filed Apr. 16, 2004; Ser. No. 10/856,340, filed May 28, 2004; Ser. No. 11/077,977, filed Mar. 11, 2005; Ser. No. 11/118,852, filed Apr. 29, 2005; 60/683,687, filed May 23, 2005; 60/723,650, filed Oct. 5, 2005 and Ser. No. 10/392,403 filed Mar. 20, 2003 the disclosures of which are all incorporated herein by reference in their entirety.

In each of the cases where stereoisomers are possible, both R and S stereoisomers are envisioned as well as any mixture of stereoisomers.

Those skilled in the art will appreciate that the invention extends to other anti-angiogenic agents within the definitions given in the claims below, having the described characteristics. These characteristics can be determined for each test compound using assays known in the art.

Administration

Surprisingly, the route of administration of the anti-angiogenic agent is not critical to achieving the desired plasma concentration and any known method of administering drug compositions to humans and/or animals can be used.

The compositions described herein can be provided as physiologically acceptable formulations using known techniques, and the formulations can be administered by standard routes. In general, the combinations can be administered by topical, oral, rectal or parenteral (e.g., intravenous, subcutaneous or intramuscular) route. In addition, the compositions can be incorporated into polymers allowing for sustained release, the polymers being implanted in the vicinity of where delivery is desired, for example, at the site of a tumor or within or near the eye, or the polymers can be implanted, for example, subcutaneously or intramuscularly or delivered intravenously or intraperitoneally to result in systemic delivery of the anti-angiogenic agent. Other formulations for controlled, prolonged release of therapeutic agents useful in the present invention are disclosed in U.S. Pat. No. 6,706,289, the disclosure of which is incorporated herein by reference.

The formulations in accordance with the present invention can be administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

The formulations include those suitable for oral, rectal, nasal, inhalation, topical (including dermal, transdermal, buccal and sublingual), vaginal, parenteral (including subcutaneous, intramuscular, intravenous, intradermal, intraocular, intratracheal, and epidural) or inhalation administration. The formulations can conveniently be presented in unit dosage form and can be prepared by conventional pharmaceutical techniques. Such techniques include the step of bringing into association the active ingredient and a pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken; i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing, in addition to the active ingredient, ingredients such as carriers as are known in the art to be appropriate.

Formulation suitable for inhalation may be presented as mists, dusts, powders or spray formulations containing, in addition to the active ingredient, ingredients such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Formulations suitable for parenteral administration include particulate preparations of the anti-angiogenic agents, including, but not limited to, low-micron, or nanometer (e.g. less than 2000 nanometers, preferably less than 1000 nanometers, most preferably less than 500 nanometers in average cross section), sized particles, which particles are comprised of the antiangiogenic agent alone or the agent in combination with accessory ingredients or the agent in a polymer for sustained release. The formulations may be presented in unit-dose or multi-dose containers, for example, sealed ampules and vials, and may be stored in freeze-dried (lyophilized) conditions requiring only the addition of a sterile liquid carrier, for example, water for injections, immediately prior to use. Extemporaneous injection solutions and suspensions may be prepared from sterile powders, granules and tablets of the kinds previously described.

Preferred unit dosage formulations are those containing a daily dose or unit, daily sub-dose, as herein above recited, or an appropriate fraction thereof, of the administered ingredient.

For oral administration, an especially preferred formulation is disclosed in U.S. patent application Ser. No. 10/392,403, filed Mar. 20, 2003, which is incorporated herein by reference in its entirety.

It should be understood that in addition to the ingredients, particularly mentioned above, the formulations of the present invention may include other agents conventional in the art having regard to the type of formulation in question, for example, those suitable for oral administration may include flavoring agents, and nanoparticle formulations (e.g. less than 2000 nanometers, preferably less than 1000 nanometers, most preferably less than 500 nanometers in average cross section) may include one or more than one excipient chosen to prevent particle agglomeration.

Indications

The invention can be used to treat any disease characterized by abnormal cell mitosis and/or abnormal or undesirable angiogenesis. Such diseases include, but are not limited to, abnormal stimulation of endothelial cells (e.g., atherosclerosis); solid tumors; blood-borne tumors, such as leukemias; tumor metastasis; benign tumors, for example, hemangiomas, acoustic neuromas, neurofibromas, trachomas, and pyogenic granulomas; vascular malfunctions; abnormal wound healing; inflammatory and immune disorders; Bechet's disease; gout or gouty arthritis; abnormal angiogenesis accompanying: rheumatoid arthritis; skin diseases, such as psoriasis; diabetic retinopathy, and other ocular angiogenic diseases, such as retinopathy of prematurity (retrolental fibroplasia), macular degeneration, corneal graft rejection, neovascular glaucoma; liver diseases and Oster Webber Syndrome (Osler-Weber Rendu disease).

Other undesired angiogenesis involves normal processes including ovulation and implantation of a blastula. The compositions described above can be used as a birth control agent by reducing or preventing uterine vascularization required for embryo implantation. Thus, the present invention provides an effective birth control method when an amount of Formulae I, II, III or IV sufficient to prevent embryo implantation is administered to a female. In one aspect of the birth control method, an amount of Formulae I, II, III or IV sufficient to block embryo implantation is administered before or after intercourse and fertilization have occurred, thus providing an effective method of birth control, possibly a “morning after” method. While not wanting to be bound by this theory, it is believed that inhibition of vascularization of the uterine endometrium interferes with implantation of the blastocyte. Similar inhibition of vascularization of the mucosa of the uterine tube interferes with implantation of the blastocyte, preventing the occurrence of a tubal pregnancy. The compositions described above can also be used to block ovulation or to block menstruation (induce amenorrhea).

Diseases associated with neovascularization can be treated according to the present invention. Such diseases include, but are not limited to, ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacterial infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, scleritis, Steven-Johnson's disease, pemphigoid, radial keratotomy, and corneal graft rejection.

Other diseases associated with neovascularization can be treated according to the present invention. Such diseases include, but are not limited to, sickle cell anemia, sarcoidosis, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, and post-laser complications. Other diseases include, but are not limited to, diseases associated with rubeosis (neovascularization of the iris and the angle) and diseases caused by the abnormal proliferation of fibrovascular or fibrous tissue including all forms of proliferative vitreoretinopathy, whether or not associated with diabetes.

The present invention may also be used to treat angiogenesis-dependent cancers including, but not limited to, any one or combination of rhabdomyosarcoma, retinoblastoma, Ewing's sarcoma, neuroblastoma, and osteosarcoma. Other angiogenesis-dependent cancers treatable with the present invention include, but are not limited to, breast cancer, prostrate cancer, renal cell cancer, brain cancer, ovarian cancer, colon cancer, bladder cancer, pancreatic cancer, stomach cancer, esophageal cancer, cutaneous melanoma, liver cancer, small cell and non-small cell lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, non-Hodgkin's lymphoma, lip cancer, oral cancer, skin cancer, leukemia or multiple myeloma.

As mentioned above, another disease that can be treated according to the present invention is rheumatoid arthritis. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Other diseases that can be treated according to the present invention are hereditary hemorrhagic telangiectasia, osteoarthritis, chronic inflammation, Crohn's disease, ulcerative colitis, Bartonellosis, inflammatory or immune mediated bowel disease and acquired immune deficiency syndrome.

The present invention can be used to treat eye conditions in humans or animals, wherein the eye conditions include, but are not limited to, ocular neovascular disease, diabetic retinopathy, retinopathy of prematurity, macular degeneration, corneal graft rejection, neovascular glaucoma, retrolental fibroplasias, epidemic keratoconjunctivitis, contact lens overwear, atopic keratitis, superior limbic keratitis, pterygium keratitis sicca, myopia, chronic retinal detachment, optic pits, Terrien's marginal degeneration, hyperviscosity syndromes, chronic uveitis, chronic vitritis, presumed ocular histoplasmosis, rubeosis, retinitis, choroiditis, proliferative vitreoretinopathy, scleritis, Eales' disease, Best's disease, trachoma, or post-laser complications.

The present invention can be used to treat inflammatory or immune mediated diseases in humans or animals, wherein the inflammatory or immune mediated diseases include, but are not limited to, rheumatoid arthritis, osteoarthritis, ulcerative colitis, Crohn's disease, Mooren's ulcer, arthritis, sarcoidosis, inflammatory or immune mediated bowel disease, systemic lupus, Wegener's syndrome, Stevens-Johnson's disease, Behcet's disease, pemphigoid, Lyme's disease, asthma or acquired immune deficiency syndrome.

The present invention can be used to treat infectious diseases in humans or animals, wherein the infectious diseases include, but are not limited to syphilis, a bacterial infection, a Mycobacterial infection, a bacterial ulcer, a fungal ulcer, a Herpes simplex infection, a Herpes zoster infection, a protozoan infection, a Bartonellosis infection, or toxoplasmosis.

The present invention can be used to treat blood or blood vessel diseases in humans or animals, wherein the blood or blood vessel diseases include, but are not limited to, vein occlusion, artery occlusion, carotid obstructive disease, polyarteritis, atherosclerosis, Osler-Weber-Rendu disease, sickle cell anemia, leukemia, acute or chronic neoplastic disease of the bone marrow, hemangiomas, hereditary hemorrhagic telangiectasia, disease of the bone marrow, anemia, impaired blood clotting or enlargement of the lymph nodes, liver, or spleen. The present invention can also be used to treat chronic neoplastic disease of the bone marrow, wherein those diseases include, but are not limited to, multiple myeloma and myelo dysplastic syndrome.

The present invention can be used to treat skin conditions in humans or animals, wherein the skin conditions include, but are not limited to, abnormal wound healing, acne rosacea, chemical burns of the skin, dermatitis or psoriasis.

In addition, the invention can be used to treat a variety of post-menopausal symptoms, osteoporosis, cardiovascular disease, myocardial angiogenesis, plaque neovascularization, hemophiliac joints, angiofibroma, wound granulation, intestinal adhesions, scleroderma, hypertrophic scars; i.e., keloids. They are also useful in the treatment of diseases that have angiogenesis as a pathologic consequence, such as cat scratch disease, and Helicobacter pylori ulcers. The invention can also be used to treat Alzheimer's disease, to reduce the incidence of stroke, and as an alternative to prior estrogen replacement therapies. The compounds of the present invention can work by estrogenic and non-estrogenic biochemical pathways.

Additionally, the compounds of the present invention can be used to treat endometriosis. Endometriosis is the abnormal growth of endometrial cells; the same cells that line the uterus that are shed monthly in the menstrual process. Wayward endometrial cells can position themselves in the lower abdomen on areas such as the cul-de-sac, the recto-vaginal septum, the stomach, the fallopian tubes, the ovaries, and the bladder. During menstruation, the normal uterine lining is sloughed off and expelled through the vagina, but transplanted endometrial tissue has no means of exiting the body; instead the endometrial tissue and cells adhere and grow where positioned. The results are internal bleeding, inflammation, and scarring. One of the serious consequences of endometrial scarring is infertility. The endometrial growths are generally not malignant or cancerous. Among other complications, the growths can rupture and can spread the endometriosis to new areas of the lower abdomen. Endometriosis is a progressive disease. The growths or lesions are first seen as clear vesicles, then become red, and finally progress to black lesions over a period of seven to ten years.

Pharmaceutical Preparations

Also contemplated by the present invention are implants or other devices comprised of the compounds or drugs of Formulae I, II, III or IV, or prodrugs thereof, or other anti-angiogenic agents included by reference where the drug or prodrug is formulated in a biodegradable or non-biodegradable polymer for sustained release. Non-biodegradable polymers release the drug in a controlled fashion through physical or mechanical processes without the polymer itself being degraded. Biodegradable polymers are designed to gradually be hydrolyzed or solubilized by natural processes in the body, allowing gradual release of the admixed drug or prodrug. The drug or prodrug can be chemically linked to the polymer or can be incorporated into the polymer by admixture. Both biodegradable and non-biodegradable polymers and the process by which drugs are incorporated into the polymers for controlled release are well known to those skilled in the art. Examples of such polymers can be found in many references, such as Brem et al., J. Neurosurg 74: pp. 441-446 (1991). These implants or devices can be implanted in the vicinity where delivery is desired, for example, at the site of a tumor or a stenosis, or can be introduced so as to result in systemic delivery of the agent.

Because anything not formed in the body as a natural component may elicit extreme and unexpected responses, such as blood vessel closure due to thrombus formation or spasm, and because damage to blood vessels by the act of insertion of a vascular stent may be extreme and unduly injurious to the blood vessel surface, it is prudent to protect against such events. Restenosis is a re-narrowing or blockage of an artery at the same site where treatment, such as an angioplasty or stent procedure, has already taken place. If restenosis occurs within a stent that has been placed in an artery, it is technically called “in-stent restenosis,” the end result being a narrowing in the artery caused by a build-up of substances that may eventually block the flow of blood. The compounds that are part of the present invention are especially useful to coat vascular stents to prevent restenosis. The coating should preferably be a biodegradable or non-biodegradable polymer that allows for a slow release of a compound of the present invention thereby preventing the restenosis event.

The present invention also relates to conjugated prodrugs and uses thereof. More particularly, the invention relates to conjugates of steroid compounds, such as compounds of Formulae I, II, III or IV, and the use of such conjugates in the prophylaxis or treatment of conditions associated with enhanced angiogenesis or accelerated cell division, such as cancer, and inflammatory conditions, such as asthma and rheumatoid arthritis and hyperproliferative skin disorders including psoriasis. The invention also relates to compositions including the prodrugs of the present invention and methods of synthesizing the prodrugs.

In one aspect, the present invention provides a conjugated prodrug of an estradiol compound, preferably compounds of Formulae I, II, III or IV, conjugated to a biological activity modifying agent.

Alternatively, the conjugated prodrug according to the present invention includes the compounds of Formulae I, II, III or IV conjugated to a peptide moiety.

The incorporation of an estradiol compound, such as the compounds of Formulae I, II, III or IV, into a disease-dependently activated pro-drug enables significant improvement of potency and selectivity of this anti-cancer and anti-inflammatory agent.

In addition to the compounds of the present invention, the pharmaceutical composition of this invention may also contain, or be co-administered (simultaneously or sequentially) with, one or more pharmacological agents of value in treating one or more disease conditions referred to hereinabove.

A person skilled in the art will be able by reference to standard texts, such as Remington's Pharmaceutical Sciences 17th edition, to determine how the formulations are to be made and how these may be administered.

In a further aspect of the present invention there is provided use of compounds of Formulae I, II, III or IV, or prodrugs thereof, according to the present invention for the preparation of a medicament for the prophylaxis or treatment of conditions associated with angiogenesis or accelerated cell division or inflammation.

In a further aspect of the present invention there is provided a pharmaceutical composition comprising compounds of Formulae I, II, III or IV, or prodrugs thereof, according to the present invention, together with a pharmaceutically acceptable carrier, diluent or excipient.

The pharmaceutical composition can be used for the prophylaxis or treatment of conditions associated with angiogenesis or accelerated cell division or inflammation.

In a still further aspect of the present invention there is provided a method of prophylaxis or treatment of a condition associated with angiogenesis or accelerated or increased amounts of cell division, hypertrophic growth or inflammation, said method including administering to a patient in need of such prophylaxis or treatment an effective amount of compounds of Formulae I, II, III or IV, or prodrugs thereof, according to the present invention, as described herein. It should be understood that prophylaxis or treatment of said condition includes amelioration of said condition.

Pharmaceutically acceptable salts of the compounds of the Formulae I, II, III or IV, or the prodrugs thereof, can be prepared in any conventional manner, for example from the free base and acid. In vivo hydrolysable esters, amides and carbamates and other acceptable prodrugs of Formulae I, II, III or IV can be prepared in any conventional manner.

The present invention includes compositions and methods for treating mammalian disease characterized by pathogenic angiogenesis by administering compounds of Formulae I, II, III or IV. The 2-methoxyestradiol, and analogs and/or derivatives thereof, are modified at the 2-, 3- and 17-positions or combinations thereof. Combinations which are physically impossible are not contemplated by this invention, such as a carbon atom containing 5 bonds.

100% pure isomers are contemplated by this invention; however a stereochemical isomer (labeled as α or β, or as R or S) may be a mixture of both in any ratio, where it is chemically possible by one skilled in the art. Also contemplated by this invention are both classical and non-classical bioisosteric atom and substituent replacements, such as are described by Patani and Lavoie (“Bio-isosterism: a rational approach in drug design” Chem. Rev. (1996) p. 3147-3176) and are well known to one skilled in the art. Such bioisosteric replacements include, for example, but are not limited to, substitution of ═S or ═NH for ═O.

A particularly preferred formulation useful in the present invention is a nanoparticulate liquid suspension of 2-methoxyestradiol disclosed in U.S. patent application Ser. No. 10/392,403, filed Mar. 20, 2003 (the disclosure of which is incorporated herein by reference). This formulation is available from EntreMed, Inc., Rockville, Md., under the designation Panzem® NCD.

Improved Estradiol Derivative Synthesis

Known compounds that are used in accordance with the invention and precursors to novel compounds according to the invention can be purchased, e.g., from Sigma Chemical Co., Steraloids or Research Plus. Other compounds according to the invention can be synthesized according to known methods from publicly available precursors.

The analogs and/or derivatives of estradiol can be prepared according to known methods, such as those described in U.S. Pat. No. 6,136,992 and U.S. patent application Ser. No. 11/077,977, filed Mar. 11, 2005 (both of which are incorporated herein by reference); Siya Ram et al., 2-Alkoxy estradiols and derivatives thereof (incorporated herein by reference); Cushman et al., J. Med. Chem. (1995) 38, 2041-2049; Cushman, et. al., J. Med. Chem. (1997) 40, 2323-2334; U.S. Pat. No. 6,448,419 (2002); Paaren et al., Wang et al, Synthetic Comm 1998, 28, 4431, Cushman et al., J. Med. Chem. 1995, 38, 2041, Cushman et al., J. Med. Chem. 2002, 45, 4748), or other publications, incorporated herein by reference.

### Experimental Data

The following Examples refer to compounds of the following general Formulae:

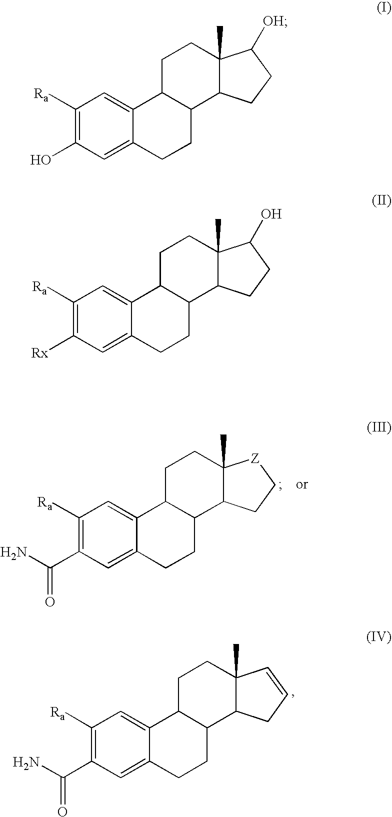

wherein Rₐ is selected from —OCH₃, —OCH₂CH₃, —CH₃, —CH₂CH₃, or —CCCH₃; Rₓ is selected from —NH₂, —F, —Cl, —Br, —CH═CH₂, —NH—CHO, —O-sulfamate; and Z is selected from >C(H₂), >C(H)—CH₃, >C═CH₂, >C═CHCH₃ (cis or trans), >C═O, >C(H)—OH, >C(H)—O-alkyl, >C(H)—O-sulfamate, wherein alkyl is a linear, branched and/or cyclic hydrocarbon chain comprising 1 to 10 carbons. Preferred species from the foregoing genus that are useful in the present invention include, but are not limited to, the compounds shown in Table 1.

TABLE 1

| | | |
|---|---|---|
| 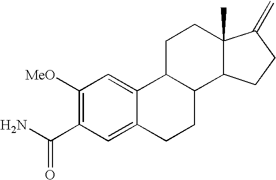 | 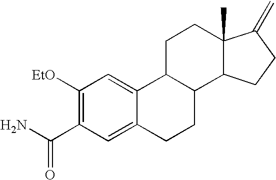 | 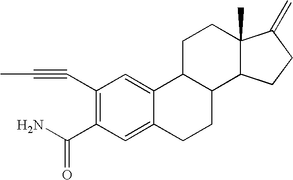 |
| 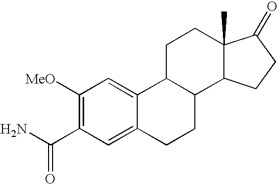 | 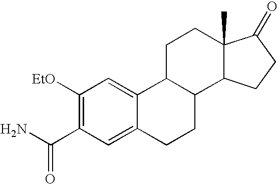 | 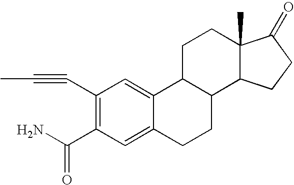 |
| 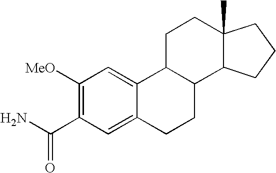 | 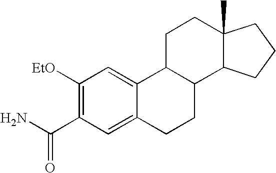 | 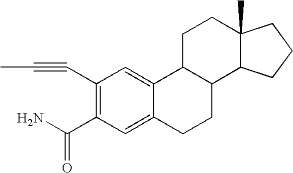 |
| 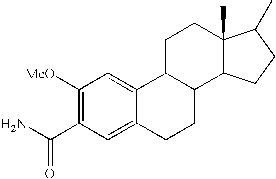 | 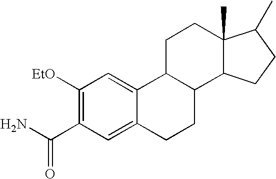 | 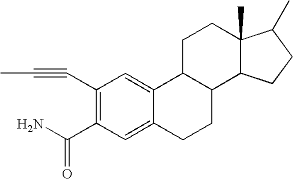 |
| 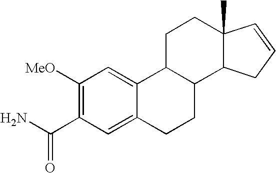 | 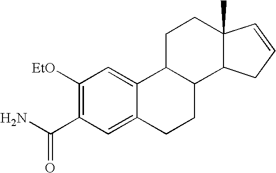 | 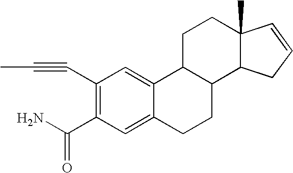 |
| 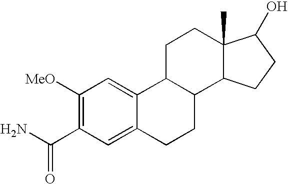 | 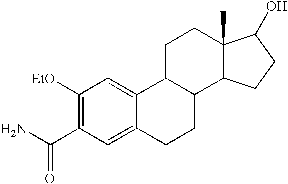 | 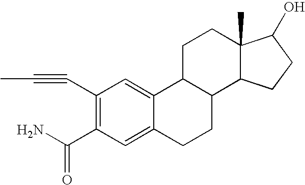 |
| 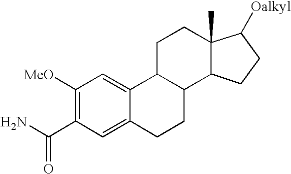 | 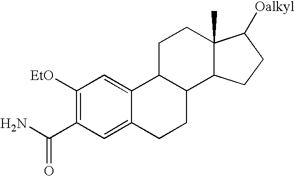 | 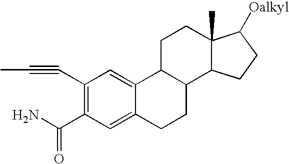 |
| 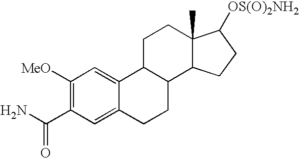 | 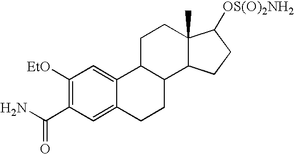 | 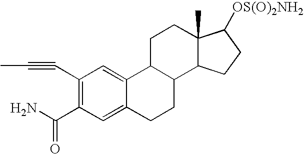 |
| 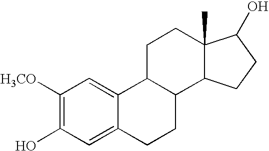 | | |

For the compounds shown in Table 1 above, alkyl is defined as a linear, branched and/or cyclic (or combinations thereof) hydrocarbon chain, which can be saturated or unsaturated, comprising 1 to 10 carbons.

Each of the foregoing compounds from Table 1 has anti-mitotic properties, anti-angiogenic properties, anti-tumor properties, anti-inflammatory properties, or combinations thereof.

A pharmacodynamic paradigm relating 2ME2 pharmacokinetics to activity in vivo was developed. Dose-response relationships were established for 2ME2 following administration by (a) i.v. bolus administration, (b) oral gavage, (c) i.p. bolus injection, (d) i.p. dose fractionation, (e) continuous i.p. infusion, or (f) drinking water ad libitum in animal models, including an experimental pulmonary metastatic Lewis lung carcinoma model, primary tumor cell xenograft models, and a model of rheumatoid arthritis. Plasma 2ME2 concentration was temporally assessed by GC-MS or LC-MS/MS methodology, and pharmacokinetic parameters were defined at effective doses.

### EXAMPLE I

These studies assess the effect of route of administration on the anti-tumor activity of 2ME2 in an orthotopic model of murine lung cancer. Three days following the intravenous injection of Lewis lung carcinoma (“LLC”), groups of ten mice were treated with 2ME2 (200 mg/kg) once every 24 hours (q.d.) by either oral gavage (p.o.) or intraperitoneal (i.p.) injection. Approximately equivalent inhibition of pulmonary metastases was observed whether 2ME2 was administered p.o. or by i.p. injection (T/C=0.28 and 0.17, respectively, p<0.05 compared to control).

### EXAMPLE II

To determine the anti-tumor effect of exposure to transient high levels of 2ME2, a series of in vivo and in vitro experiments were performed. 2ME2 was formulated as a full solubilized preparation using water-soluble excipients.

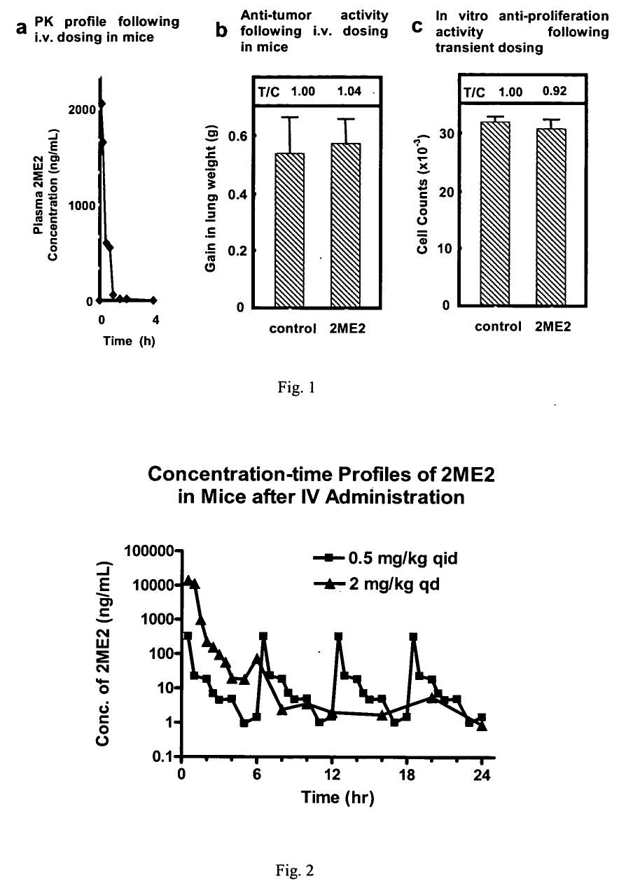

FIG. 1, Panel A: 2ME2 solution was injected into the tail vein of male C57BL/6 mice (5-6 wks, Jackson Labs). Blood samples were collected over 24 hours, and plasma separated for analysis. Levels of 2ME2 were determined using a specific GCMS assay. Panel A shows the plasma PK profile resulting from i.v. administration of 2ME2.

FIG. 1, Panel B: 2.5×10⁵ Lewis lung carcinoma cells in a volume of 0.2 ml were i.v. injected into the lateral tail vein of C57BL/6 male mice, 5-6 weeks of age (Jackson Labs). Treatment was initiated three days later by daily i.v. injection with 2ME2. Following the final day of therapy, mice were sacrificed, lungs removed, washed in phosphate buffered saline and weighed. Results are expressed as lung weight gain in treated versus control animals.

FIG. 1, Panel C: We designed a “wash-out” in vitro proliferation study to mimic the exposure profile following i.v. dosing. The LLC cell line was plated in 96-well plates. After 24 hours, medium containing 3microM 2ME2 was added. Cells were exposed to 3microM 2ME2 for 2 hours. The medium was then removed and replaced with medium alone (medium: DMEM/F12 (1:1)+10% FBS+2mM L-Glutamine). Panel C shows the poor anti-proliferative effect of transient exposure of LLC cells to 2ME2.

FIG. 1 shows that short-term exposure to transiently high levels of 2ME2 following i.v. dosing did not result in significant anti-tumor activity in vivo. This parallels the data showing that transient exposure to 2ME2 in vitro did not result in much anti-proliferative activity.

### EXAMPLE III

Dose-dependent inhibition of pulmonary metastases was observed following daily bolus administration of 2ME2. Fractionating a bolus q.d. injection (once every 24 hours, 2 mg/injection, 100 mg/kg/day) into four smaller, equal doses (q6hr, 0.5 mg/injection, 100 mg/kg/day) improved the antitumor activity of 2ME2 (T/C=0.30 and 0.05, respectively, p<0.05).

Non-tumor bearing male C57BL/6 mice (6 weeks of age) were treated for 7 days with i.p. injections of 2ME2 at 2 mg/day (100 mg/kg/day) using either a 2 mg q.d. or 0.5 mg q.i.d. schedule comparable to that used for tumor therapy. On day 7, plasma samples were collected at selected time points, and plasma unconjugated 2ME2 levels were determined using LC-MS/MS methodology. The results are shown below in Table 2.

TABLE 2

| | | | | AUC₀₋₆ | AUC₀₋₂₄ | |
|---|---|---|---|---|---|---|
| Dose | Cmax | Tmax | Cmin | (ug/ | (ng/ | T½ |
| (mg/kg) | (ng/mL) | (hr) | (ng/mL) | mL*hr) | mL*hr) | (hr) |
| 0.5 q.d. | 325 | 0.5 | 1.46 | 206.6 | 826.5^{a} | 1.1 |
| 0.5 q.i.d. | 326.09 | 18.5 | 1.50 | 206.6 | 836.1 | 1.1 |
| 2.0 q.d. | 14070 | 0.5 | 0.86 | 13445.1 | 13570.9 | 3.2 |

^{a}= AUC₀₋₆*4

The data for 0.5 mg q.i.d. over the first six hours was used to model steady state levels over a 24 hr. time period as shown in FIG. 2. Pharmacokinetic parameters were determined by noncompartmental analysis using WinNonLin (Pharsight, Mountain View, Calif.). Pharmacokinetic studies comparing bolus q.d. treatment versus q6hr dosing supports the previous observations concluding that transient high initial peak concentrations of 2ME2 are not accounting for maximal antitumor activity; however, a parameter related to “time over threshold” is suggested for the activity of 2ME2 in this model (see FIG. 2).

### EXAMPLE IV

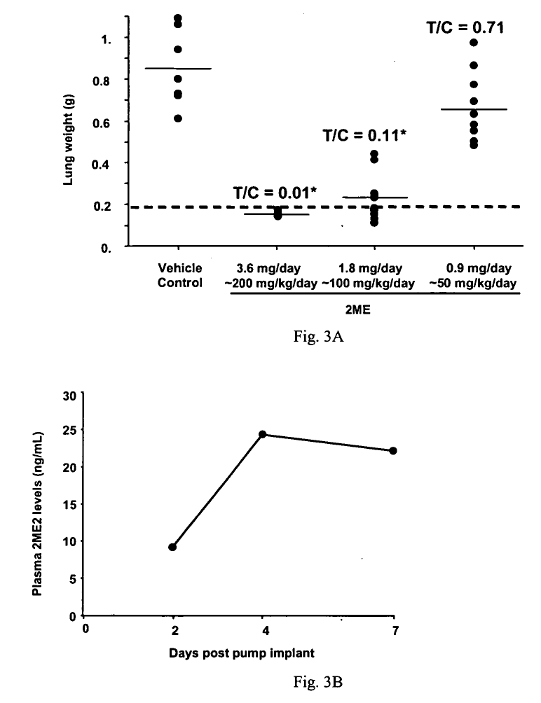

In further support of this paradigm, a significant inhibition of pulmonary metastases was obtained in tumor-bearing mice given 2ME2 by continuous infusion using Alzet pumps. Three days after injection of LLC cells, therapy was initiated i.p. with either vehicle control or with 2ME2 delivered through 7-day osmotic pumps (see FIG. 3A). The daily target levels of 2ME2 were calculated assuming an average pump rate of 1.0 mL/h. To achieve a target level of 200 mg/kg/day, the concentration of 2ME2 in the osmotic pumps was prepared at 150 mg/mL. Asterisks indicate groups statistically different from vehicle control (α=0.05) as shown in A. To determine the PK profile of 2ME2 using this delivery scheme, non-tumor bearing male C57BL/6 mice (6 weeks of age) were treated i.p. for up to 7 days with 2ME2 at 1.8 mg/day (˜100 mg/kg/day) using 7-day osmotic pumps. On days 2, 4, and 7, plasma samples were collected and plasma 2ME2 levels were determined as described above (see FIG. 3B).

Multiple formulations of 2ME2 have been used in Alzet pumps to create continuous infusion effects. These formulations include, but are not limited to, 2ME2 250 mg/mL in 100% PEG 600; 2ME2 130 mg/mL in 50% PEG 400/50% DMSO; 2ME2 22 mg/mL in 100% PEG 400; 2ME2 12 mg/mL in 45% cyclodextrin/25% PVP; 2ME2 9.5 mg/mL in 45% cyclodextrin; and, 2ME2 8.5 mg/mL in 45% cyclodextrin. Cyclodextrins which can be used include, but are not limited to, unmodified sulfobutyl, hydroxypropyl-gamma- and hydroxypropyl-beta-cyclodextrins. Continuous exposure of 2ME2 using Alzet osmotic pumps generates significant antitumor activity in the LLC experimental metastasis model. At a calculated 2ME2 dose of ˜100 mg/kg/day, at which growth inhibition of pulmonary metastases was 89%, plasma exposure levels ranged from 9 to 25 ng/mL.

### EXAMPLE V

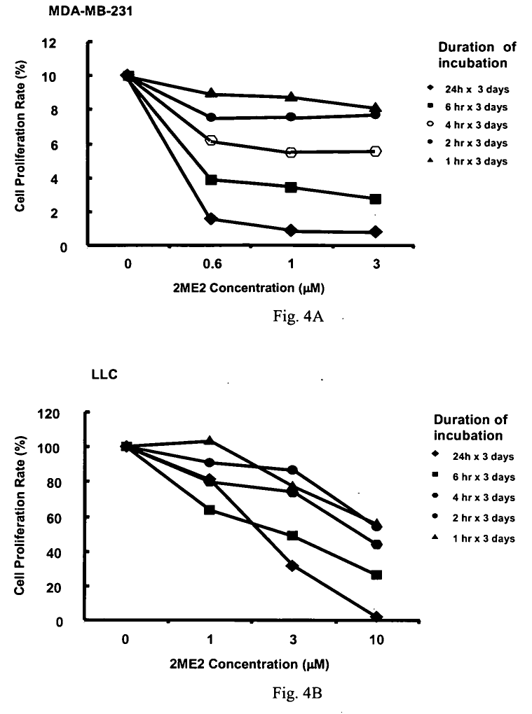

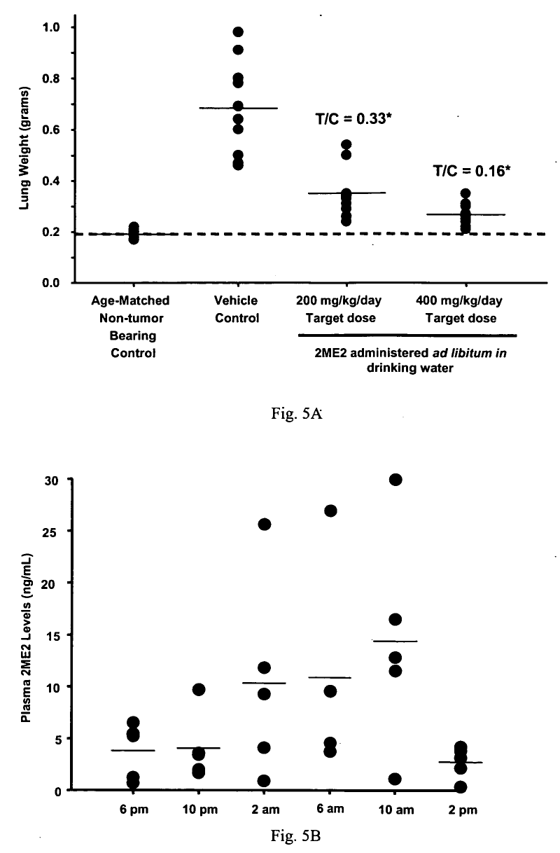

To mimic the effect of cyclic levels of 2ME2 seen after daily dosing, and to compare this to the effects of continuous exposure to 2ME2, we carried out a series of wash-out in vitro anti-proliferation studies. Tumor cell lines (Lewis lung carcinoma or MDA-MB-231) were treated in vitro for varying times with varying concentrations of 2ME2. Over the course of a three-day study, on each study day the cells were exposed to 2ME2 for varying times (1, 2, 4, 6, or 24 hours) at the indicated concentrations. The medium was then removed and replaced with medium without 2ME2 for the remainder of each 24-hour period. Anti-proliferative activity was determined using a BrdU proliferation assay according to the manufacturer's instructions after the third day of treatment (see FIGS. 4A and 4B).

Short-term incubation of cells with 2ME2, even at high concentrations, does not result in much anti-proliferative activity in vitro. This finding parallels the data indicating that short-term exposure to transiently high levels of 2ME2 following i.v. dosing did not result in significant anti-tumor activity in vivo.

Continuous exposure of tumor cells to 2ME2 results in maximal inhibition of proliferation in vitro, in parallel to the data indicating improved anti-tumor activity after substantially continuous exposure to 2ME2 in vivo.

### EXAMPLE VI

In further support of this paradigm, a significant inhibition of pulmonary metastases was obtained in tumor-bearing mice given 2ME2 ad libitum access through their drinking water. In this study, a T/C=0.16 was achieved at plasma 2ME2 concentrations ranging from 3-15 ng/mL over a 24 hr period (see FIGS. 5A and 5B).

Using relevant PK data for any given formulation (in this case an ad libitum drinking water formulation) in a human or animal, it is possible for one skilled in the art to devise a dosing strategy—a combination of dose level and dose frequency—which will result in substantially continuous maintenance of the plasma level of the agent within the desired concentration range and so maximize the pharmacological activity, in this case anti-tumor activity.

### EXAMPLE VII

In an additional rat tumor model (orthotopic H2122 NSCLC), plasma 2ME2 concentrations ranging from 3-17 ng/mL over a 24 hour period following oral gavage resulted in a 2.5-fold increase in median survival time as compared to diluent-treated controls (p<0.05). Female athymic nude rats were injected i.t. with H2122 human NSCLC cells (1×10⁶ in 0.2 mL). Seven days later, daily therapy was initiated p.o. with either vehicle control or 2ME2 at 10 or 60 or 300 mg/kg/d (n=15 rats/group). Animals were monitored closely for signs of morbidity and median survival times (MST) were determined. The study was terminated 50 days post tumor cell injection. In a separate cohort of non-tumor-bearing rats, plasma 2ME2 concentrations were determined at selected time points following a single oral gavage at 60 and 300 mg/kg. The PK data in Table 3 represents the range of 2ME2 concentrations in a 24 hour period. Although this strain and sex rat has quite different PK parameters to male nude mice, as one example comparison, a similar relationship between PK (substantially continuous exposure over a threshold) and pharmacodynamics (anti-tumor activity) was seen in this study. Using relevant PK data for any given formulation in a human or animal, it is possible for one skilled in the art to devise a dosing strategy—a combination of dose level and dose frequency—which will result in substantially continuous maintenance of the plasma level of the agent within the desired concentration range and so maximize the pharmacological activity, in this case anti-tumor activity. The results are shown below in Table 3.

TABLE 3

| | Median | | 2ME2 Plasma |
|---|---|---|---|
| | Survival | % Survival | Concentration Range |
| Treatment Group | Time (days) | on Day 50 | (ng/mL) |
| Vehicle Control | 17 | 0 | — |
| 2ME2 10 mg/kg/d | 18.5 | 13 | not determined |
| 2ME2 60 mg/mk/d | 26 | 20 | 1-10 |
| 2ME2 300 mg/kg/d | 43 | 47 | 3-17 |

### EXAMPLE VIII

A significant inhibition of osteolytic lesions was obtained in a rat collagen-induced arthritis model after -treatment with 2ME2 by continuous infusion using Alzet pumps. In this study, groups of rats (n=10) began treatment on day 10 following collagen-induced arthritis with (a) vehicle alone, (b) 2ME2 100 mg/kg p.o. q.d., (c) 50 mg/kg p.o. b.i.d., (d) or 60 mg/kg s.c. by continuous infusion using Alzet pumps. The following radiographic scores were obtained on day 28: vehicle 4.3±0.19; 2ME2 100 mg/kg p.o. q.d. 2.88±0.21; 2ME2 50 mg/kg p.o. b.i.d 2.11±0.18; and 2ME2 60 mg/kg s.c. continuous infusion 1.5±0.18. These data further support the pharmacodynamic paradigm of time-above-threshold to maximize the activity of 2ME2. Using relevant PK data for any given formulation in a human or animal, it is possible for one skilled in the art to devise a dosing strategy—a combination of dose level and dose frequency—which will result in substantially continuous maintenance of the plasma level of the agent within the desired concentration range and so maximize the pharmacological activity, in this case anti-arthritic or anti-inflammatory activity.

### EXAMPLE IX

To mimic the effect of cyclic levels of ENMD-1198 seen after daily dosing, and to compare this to the effects of continuous exposure to ENMD-1198, we carried out a series of wash-out in vitro anti-proliferation studies.

The structure of ENMD-1198 is:

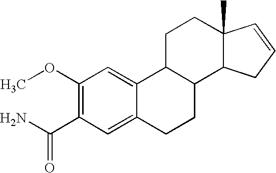

Tumor cell lines (Lewis lung carcinoma or MDA-MB-231) were treated in vitro for varying times with varying concentrations of ENMD-1198. Over the course of a three-day study, on each study day the cells were exposed to ENMD-1198 for varying times (1, 2, 4, 6, or 24 hours) at the indicated concentrations. The medium was then removed and replaced with medium without ENMD-1198 for the remainder of each 24-hour period. For the 24-hour exposure wells, the medium was changed every day to provide 72 hours continuous exposure. Anti-proliferative activity was determined by cell counts after the third day of treatment.

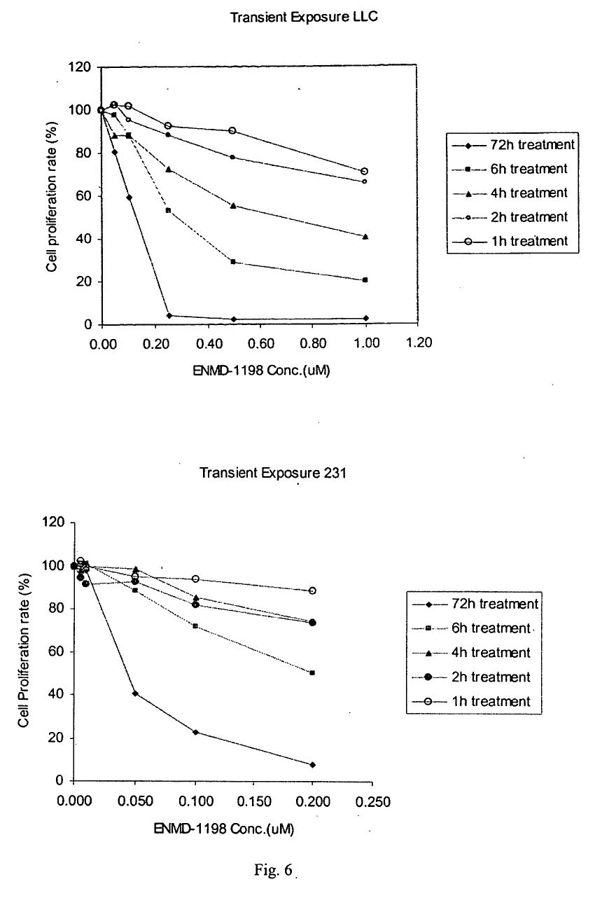

Short-term incubation of cells with ENMD-1198, even at high concentrations, does not result in much anti-proliferative activity in vitro. This finding parallels the data indicating that short-term exposure to transiently high levels of 2ME2 did not result in significant anti-proliferative activity in vitro. Continuous exposure of tumor cells to ENMD-1198 results in maximal inhibition of proliferation in vitro (see FIG. 6).

### EXAMPLE X

Multiple formulations of 2ME2 are available that can be employed to achieve the desired time-above-threshold range. Data in Table 4 indicate selected PK parameters achieved for a wide variety of formulations delivered orally at a selected dose. The formulation approaches used to generate PK data after oral dosing include but are not limited to nanometer-sized crystals, amorphous precipitates of controlled particle size, non-aqueous vehicles, and, spray-dried precipitates with co-polymers. Using the data in this Table, it is possible for one skilled in the art to devise a dosing strategy—a combination of dose level and dose frequency—which will result in substantially continuous maintenance of the plasma level of the agent within the desired concentration range and so maximize the desired activity. The results are shown in Table 4.

TABLE 4

| | Cmax | C_{4 hr} | AUC_{T} |
|---|---|---|---|
| Formulation | (nM) | (nM) | (nM*hr) |
| E1 | 96.03 | 29.8 | 1304.31 |
| E2 | 158.94 | 16.61 | 310.44 |
| B1 | 420 | 35.4 | 734.75 |
| B2 | 296.67 | 20.86 | 1117.31 |
| B3 | 417 | 149.1 | 1248.18 |
| D1 | 52.8 | 28.65 | 523.4 |
| D2 | 74.58 | 9.04 | 165.92 |
| A1 | 188.43 | 35.97 | 602.96 |
| A2 | 271.59 | 36.30 | 489.8 |
| C1 | 603.9 | 49.83 | 1521.02 |
| C2 | 1009.8 | 38.61 | 1134.31 |
| N1 | 3180.15 | 25.27 | 2006.72 |
| N3 | 1252.08 | 23.58 | 390.23 |
| N4 | 84.92 | 22.24 | 522.15 |
| N5 | 18.48 | 8.85 | 216.68 |
| N6 | 869.13 | 22.41 | 1013.48 |
| N7 | 432.9 | 32 | 654.51 |
| N8 | 49.28 | 49.28 | 591.56 |
| N9 | 128.54 | 70.26 | 817.94 |
| N10 | 2111.22 | 18.35 | 1729.69 |
| N11 | 1431.9 | 104.23 | 2157.44 |
| N12 | 999 | 58.61 | 1318.57 |
| N13 | 919.08 | 123.54 | 1971.71 |
| N14 | 1984.68 | 212.79 | 2780.18 |

The invention described here is that maximizing anti-angiogenic, anti-proliferative, anti-mitotic, or anti-inflammatory activity or combinations thereof for the described agents can be achieved by substantially maintaining plasma drug levels within a specified target range for a desired period of time. The examples given are not limiting. Other agents described herein will have a specific “therapeutic window” which maximizes clinical benefit while minimizing any toxicities or other deleterious activities or effects. The Examples above provide several methods for determining the range of plasma levels and length of exposure which are appropriate for each agent to achieve maximal anti-angiogenic, anti-proliferative, anti-mitotic, or anti-inflammatory activity or combinations thereof. One skilled in the art can use these Examples and apply them to the claimed agents to determine the specific target concentration range and length of time over threshold required for maximal activity.

Continuous exposure can be achieved by the use of sustained release drug delivery systems including implanted or parenteral polymers or slow-release or pulsed-release oral formulations. It is also well known to those skilled in the art that maintaining plasma exposure over a threshold level can also be achieved by matching a drug/formulation combination to a dose level and dosing schedule. These procedures are known in the field by various names including “dosing up” or “dosing to steady state”. As an example, an oral formulation which results in a short half-life of drug levels in plasma can be dosed at a higher level or dosed more frequently to maintain plasma levels above a desired threshold, such dose and dosing schedule chosen based on mathematical modeling of the pharmacokinetic profile of the formulation using published formulas or calculations or commercially available software programs.

As another example, a formulation which results in extended exposure at high levels can be dosed on a less frequent schedule, such dose and dosing schedule chosen based on mathematical modeling of the pharmacokinetic profile of the formulation using published formulas or calculations or commercially available software programs. As another example, a formulation which results in extended exposure at low levels can be “dosed up” using a more frequent dosing schedule until the plasma levels are shown to be or are predicted to be within the defined range, such dose and dosing schedule chosen based on mathematical modeling of the pharmacokinetic profile of the formulation using published formulas or calculations or commercially available software programs known to those skilled in the art.

The Examples above indicate how dose level and dosing schedule can be adjusted to match the exposure characteristics of the formulation, such that plasma levels of the anti-angiogenic agent can be maintained within the desired range for the specified period of time in each dosing period.

All of the publications mentioned herein are hereby incorporated by reference in their entireties. The above examples are merely demonstrative of the present invention, and are not intended to limit the scope of the invention or the appended claims.

We claim:

1. A method of administering a therapeutic agent to a human or animal comprising administering to the human or animal an amount of the therapeutic agent selected from 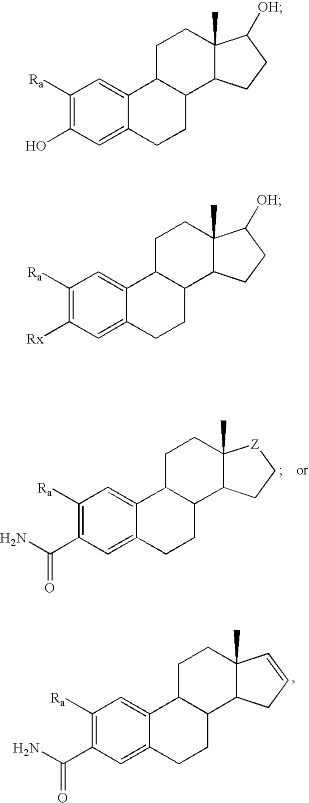
wherein Rₐ is selected from —OCH₃, —OCH₂CH₃, —CH₃, —CH₂CH₃, or —CCCH₃; Rₓ is selected from —NH₂, —F, —Cl, —Br, —CH═CH₂, —NH—CHO, —O-sulfamate; and Z is selected from >C(H₂), >C(H)—CH₃, >C═CH₂, >C═CHCH₃ (cis or trans), >C═O, >C(H)—OH, >C(H)—O-alkyl or >C(H)—O-sulfamate wherein alkyl is a linear, branched and/or cyclic hydrocarbon chain containing 1-10 carbons such that a plasma concentration of the therapeutic agent in the human or animal is substantially continuously maintained above approximately 1 ng/mL.

2. The method of claim 1, wherein the plasma concentration of the therapeutic agent is between approximately 1 ng/mL and 300 ng/mL.

3. The method of claim 1, wherein the plasma concentration of the therapeutic agent is between approximately 2 ng/mL and 150 ng/mL.

4. The method of claim 1, wherein the plasma concentration of the therapeutic agent is between approximately 3 ng/mL and 100 ng/mL.

5. The method of claim 1, wherein the plasma concentration of the therapeutic agent is between approximately 3 ng/mL and 50 ng/mL.

6. The method of claim 1, wherein the administration of the therapeutic agent is oral, parenteral, transdermal, topical, intravenous, subcutaneous, intramuscular, intradermal, ophthalmic, epidural, intratracheal, sublingual, buccal, rectal, vaginal, nasal or inhalation.

7. The method of claim 1, wherein the therapeutic agent is administered in a composition comprising an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

8. The method of claim 1, wherein the therapeutic agent is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

9. A method of treating a disease in a human or animal comprising administering to the human or animal an amount of a therapeutic agent selected from 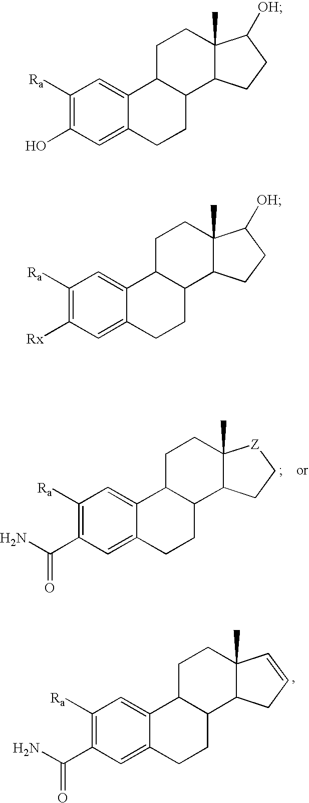
wherein Rₐ is selected from —OCH₃, —OCH₂CH₃, —CH₃, —CH₂CH₃, or —CCCH₃; Rₓ is selected from —NH₂, —F, —Cl, —Br, —CH═CH₂, —NH—CHO, —O-sulfamate; and Z is selected from >C(H2), >C(H)—CH₃, >C═CH₂, >C═CHCH₃ (cis or trans), >C═O, >C(H)—OH, >C(H)—O-alkyl or >C(H)—O-sulfamate wherein alkyl is a linear, branched and/or cyclic hydrocarbon chain containing 1-10 carbons such that a plasma concentration of the therapeutic agent in the human or animal is substantially continuously maintained above approximately 1 ng/mL.

10. The method of claim 9, wherein the plasma concentration of the therapeutic agent is between approximately 1 ng/mL and 300 ng/mL.

11. The method of claim 9, wherein the plasma concentration of the therapeutic agent is between approximately 2 ng/mL and 150 ng/mL.

12. The method of claim 9, wherein the plasma concentration of the therapeutic agent is between approximately 3 ng/mL and 100 ng/mL.

13. The method of claim 9, wherein the plasma concentration of the therapeutic agent is between approximately 3 ng/mL and 50 ng/mL.

14. The method of claim 9, wherein the administration of the therapeutic agent is oral, parenteral, transdermal, topical, intravenous, subcutaneous, intramuscular, intradermal, ophthalmic, epidural, intratracheal, sublingual, buccal, rectal, vaginal, nasal or inhalation.

15. The method of claim 9, wherein the therapeutic agent is administered in a composition comprising an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

16. The method of claim 9, wherein the therapeutic agent is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

17. The method of claim 9, wherein the disease is associated with diabetic retinopathy, retinopathy of prematurity, corneal graft rejection, neovascular glaucoma, retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacterial infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, arthritis, rheumatoid arthritis, polyarteritis, systemic lupus, Wegener's sarcoidosis, scieritis, Stevens-Johnson's disease, radial keratotomy, macular degeneration, sickle cell anemia, sarcoidosis, pseudoxanthoma elasticum, Paget's disease, vein occlusion, artery occlusion, carotid obstructive disease, chronic uveitis, chronic vitritis, Lyme's disease, Eales' disease, Behcet's disease, myopia, optic pits, Stargardt's disease, pars planitis, chronic retinal detachment, hyperviscosity syndromes, toxoplasmosis, post-laser complications, abnormal proliferation of fibrovascular or fibrous tissue, hemangiomas, Osler-Weber-Rendu disease, solid tumors, blood-borne tumors, acquired immune deficiency syndrome, ocular neovascular disease, age-related macular degeneration, rubeosis, osteoarthritis, diseases caused by chronic inflammation, Crohn's disease, ulcerative colitis, rhabdomyosarcoma, tumors of retinoblastoma, Ewing's sarcoma, neuroblastoma, osteosarcoma, leukemia, psoriasis, atherosclerosis, pemphigoid, infections causing retinitis, infections causing choroiditis, presumed ocular histoplasmosis, Best's disease, proliferative vitreoretinopathy, Bartonellosis, acoustic neuromas, neurofibroma, trachoma, pyogenic granulomas, tumor metastasis, benign tumors, vascular malfunctions, abnormal wound healing, gout, gouty arthritis, hereditary hemorrhagic telangiectasia, post-menopausal symptoms, osteoporosis, cardiovascular disease, myocardial angiogenesis, plaque neovascularization, hemophiliac joints, angiofibroma, wound granulation, intestinal adhesions, scleroderma, keloids, or endometriosis.

18. The method of claim 9, wherein the disease is associated with angiogenesis-dependent cancers selected from breast cancer, prostrate cancer, renal cell cancer, brain cancer, ovarian cancer, colon cancer, bladder cancer, pancreatic cancer, stomach cancer, esophageal cancer, cutaneous melanoma, liver cancer, small cell and non-small cell lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, lip cancer, oral cancer, skin cancer, leukemia or multiple myeloma.

19. A method of treating a disease in a human or animal comprising administering to the human or animal an amount of a therapeutic agent having the following formula: 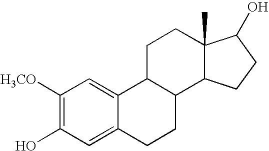
such that a plasma concentration of the therapeutic agent in the human or animal is substantially continuously maintained above approximately 1 ng/mL.

20. The method of claim 19, wherein the plasma concentration of the therapeutic agent is between approximately 1 ng/mL and 300 ng/mL.

21. The method of claim 19, wherein the plasma concentration of the therapeutic agent is between approximately 2 ng/mL and 150 ng/mL.

22. The method of claim 19, wherein the plasma concentration of the therapeutic agent is between approximately 3 ng/mL and 100 ng/mL.

23. The method of claim 19, wherein the plasma concentration of the therapeutic agent is between approximately 3 ng/mL and 50 ng/mL.

24. The method of claim 19, wherein the administration of the therapeutic agent is oral, parenteral, transdermal, topical, intravenous, subcutaneous, intramuscular, intradermal, ophthalmic, epidural, intratracheal, sublingual, buccal, rectal, vaginal, nasal or inhalation.

25. The method of claim 19, wherein the therapeutic agent is administered in a composition comprising an additive selected from an anti-oxidant, a buffer, a bacteriostat, a liquid carrier, a solute, a suspending agent, a thickening agent, a flavoring agent, a gelatin, a glycerin, a binder, a lubricant, an inert diluent, a preservative, a surface active agent, a dispersing agent, a biodegradable polymer, or any combination thereof.

26. The method of claim 19, wherein the therapeutic agent is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

27. A method of treating a disease in a human or animal comprising administering to the human or animal an amount of a therapeutic agent having the following formula: 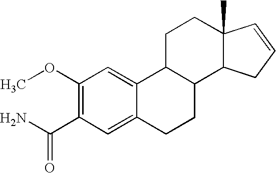
such that a plasma concentration of the therapeutic agent in the human or animal is substantially continuously maintained above approximately 1 ng/mL.

28. The method of claim 27, wherein the plasma concentration of the therapeutic agent is between approximately 1 ng/mL and 300 ng/mL.

29. The method of claim 27, wherein the plasma concentration of the therapeutic agent is between approximately 2 ng/mL and 150 ng/mL.

30. The method of claim 27, wherein the plasma concentration of the therapeutic agent is between approximately 3 ng/mL and 100 ng/mL.

31. The method of claim 27, wherein the plasma concentration of the therapeutic agent is between approximately 3 ng/mL and 50 ng/mL.

32. The method of claim 27, wherein the administration of the therapeutic agent is oral, parenteral, transdermal, topical, intravenous, subcutaneous, intramuscular, intradermal, ophthalmic, epidural, intratracheal, sublingual, buccal, rectal, vaginal, nasal or inhalation.

33. The method of claim 27, wherein the therapeutic agent is administered in a composition comprising an additive selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

34. The method of claim 27, wherein the therapeutic agent is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.
